(19) United States
(12) Patent Application Publication
(10) Pub. No.: US 2010/0300453 A1
(43) Pub. Date: Dec. 2, 2010

(54) Prophylactic Undergarment System

(76) Inventor: Kasey John Wells, Lexington, IL (US)

Correspondence Address: Kasey John Wells, 120 S. Vine Street, Lexington, IL, 61753, US

(21) Appl. No.: 12/790,825

(22) Filed: May 30, 2010

Related U.S. Application Data
(60) Provisional application No. 61/183,067, filed on Jun. 1, 2009.

(51) Int. Cl. A61F 6/04

(52) U.S. Cl. 128/844

(57) ABSTRACT

Embodiments of the present invention provide a prophylactic undergarment system made from soft, stretchable, fluid proof, stain resistant, anti-static, noise-free, high performance medical material that can be washed and reused. The system includes a condom-retaining device and is worn in conjunction with a condom during sexual activity to provide a protective barrier over the entire genital region. The system may also include a pouch for maintaining a condom in a convenient location. The undergarment system reduces skin-to-skin contact and the exchange of bodily fluids beyond typical condom coverage, which prevents the transmission of sexual diseases commonly passed or “shed” around the unprotected genital areas.

### RELATED APPLICATIONS

The present application claims priority from U.S. Provisional Application Ser. No. 61/183,067 filed Jun. 1, 2009, which is incorporated herein by reference in its entirety for all purposes.

### FIELD

The present invention relates generally to the field of sexually transmitted disease prevention and sexual enhancement. More specifically, embodiments of the present invention relate to improved systems for male prophylactic undergarments designed to be effective disease preventing and condom retaining devices with built in sexual performance enhancement mechanisms.

### BACKGROUND

One of the challenges of developing prophylactic devices is providing overall protection between the genitals, anus or other orifice and fluids secreted, infections, or diseases present during sexual activity.

In 2006, the Center of Disease Control and Prevention released information on the number of STD cases reported to CDC from the 50 states and the District of Columbia; and the results were pandemic. http://www.cdc.gov/std

“HPV infection: Approximately 20 million Americans are currently infected with HPV, and another 6.2 million people become newly infected each year. At least 50% of sexually active men and women acquire genital HPV infection at some point in their lives.”

“Herpes: Nationwide, at least 45 million people ages 12 and older, or one out of five adolescents and adults, have had genital HSV infection.”

“Gonorrhea: is a very common infectious disease. CDC estimates that more than 700,000 persons in the U.S. get new gonorrheal infections each year. Only about half of these infections are reported to CDC.”

“Chlamydia: is the most frequently reported bacterial sexually transmitted disease in the United States. An estimated 2,291,000 non-institutionalized U.S. civilians ages 14-39 are infected with Chlamydia based on the U.S. National Health and Nutrition Examination Survey.”

“Syphilis: health officials reported over 36,000 cases of syphilis in 2006, including 9,756 cases of primary and secondary (P&S) syphilis.”

“Trichomoniasis: is the most common curable STD in young, sexually active women. An estimated 7.4 million new cases occur each year in women and men.”

“HIV/AIDS: an estimated 1,106,400 adults and adolescents—living with HIV infection in the United States at the end of 2006.”

“Hepatitis A: there were an estimated 32,000 new hepatitis A virus infections in 2006.”

“Hepatitis B: In 2006, there were an estimated 46,000 new hepatitis B virus infections in the United States . . . an estimated 800,000 to 1.4 million persons have chronic hepatitis B virus infection.”

“Hepatitis C: In 2006, there were an estimated 19,000 new hepatitis C virus infections in the United States. An estimated 3.2 million persons . . . have chronic hepatitis C virus infection.”

“Molluscum Contagiosum: Overall infection rates are approximately 5% to 8% of the population. Additionally, up to 20% of people with active HIV have mulluscum contagiosum.”

“Pubic Lice/Genital Crabs: are one of the most common sexually transmitted infections with more than 3 million cases occurring in the United States every year.”

The breakdown:

| | |
|---|---|
| HPV infection: | 26,200,000 |
| Herpes: | 4,500,000 |
| Gonorrhea: | 700,000 |
| Chlamydia: | 2,291,000 |
| Syphilis: | 36,000 |
| Trichomoniasis: | 7,400,000 |
| HIV/AIDS: | 1,106,400 |
| Hepatitis A: | 32,000 |
| Hepatitis B: | 1,400,000 |
| Hepatitis C: | 3,200,000 |
| Molluscum: | 1,500,000 |
| Crabs: | 3,000,000 |
| Estimated Total: | 91,865,400 infected Americans |

This calculated total should be viewed as an approximation. It contains a combination of statistics that include: reported annual infections rates, chronic cases, and educated estimates. Some people may have more than one of the listed STDs and may have been counted multiple times. In addition, other lesser know STDs have not been listed, such as: Chacroid, Granuloma Inguinale, Lymphogranuloma Venereum, and Nongonococcal Urethritis, which affect thousands if not millions of others are not included in the totals. It should also be noted that the official numbers of reported STD cases are much lower since many people who are infected never have symptoms and/or are never reported to public health officials.

According to 2006 U.S. Population statistics, there were approximately 299,398,474 people in the U.S. Of those approximately 56,390,815 were between the ages of 0 and 13. As a result, there was an estimated 243,007,669 possibly sexually active individuals. Based on these numbers, well over ⅓ of the American population that may be sexually active is carrying one of the listed sexually transmitted diseases (in 2006), it is evident that the standard “safe sex” practices presently in use are not adequately protecting sexually active individuals against the threat of STDs.

Condoms are the most commonly used prophylactic system in the world today. While condoms are highly effective birth control devices when used properly, nevertheless there are many downfalls to this popular prophylactic. First, condoms provided limited protection to prevent the spread of STDs, because the many STDs are transferred around the unprotected genital areas beyond typical condom coverage. Second, condoms are not always readily available prior to engaging in sexual activity. Furthermore, if a condom wearer were to slightly loose his erection during sex, the condom may slip off, thereby completely compromising the condom's intended function. Despite these shortcomings, condoms have remained the prevailing prophylactic for decades. Embodiments of the present invention provide novel systems and methods designed to complement condom use. Embodiments of the invention provide unique systems and methods to effectively address and correct the deficiencies associated with condoms and provide those who are sexually active with a more comprehensive prophylactic system that provides adequate coverage and fluid proof protection over the entire genital region. The embodiments aid in providing users with a means to safeguard themselves and others from contracting and spreading STDs. Furthermore, embodiments of this novel system also comes with a built-in condom pouch that holds and protects a condom until the opportune moment arises, helps men maintain longer erections, and prevents the condom from slipping off during sexual activity

While it has been recognized that some sort of protective barrier needs to be placed over the entire genital region in order to adequately protect oneself and others from contracting or spreading STDs, still no other system provides the novel all-encompassing solution to each issue at hand. In addition, implementing this novel prophylactic system into a pair of standard everyday male underwear, improves the effectiveness of typical condom use, because it provides a convenient location for condom access and yet it conceals any noticeable exterior features that would identify it as a prophylactic device.

### SUMMARY

It is a purpose of embodiments of the invention to provide a male prophylactic undergarment system that can be inconspicuously worn as everyday underwear, while optionally providing a convenient location for concealing a condom. Embodiments of the invention are intended to compliment and correct several of the deficiencies connected to typical condom use, turning the underwear system into an effective disease preventing, condom retaining device with a built in sexual performance enhancement mechanism (i.e., a “cock-ring”) intended to prevent the spread of STDs transferred by skin to skin contact and the excretion of sexual fluids. Embodiments of the invention can be washed and reused several times. Alternatively, in one embodiment the undergarment system is disposable, or comprised from materials intended for single use. In another embodiment, the material may contain a specialized marking system that changes color over time in order to provide the wearer a visual indicator as to when the underwear would need to be replaced.

In one embodiment, a prophylactic undergarment system is provided including a flexible outer casing having front, rear and lateral aspects defining an undergarment with a flexible waistband. The front aspect has two exterior panels, equidistantly positioned and centered over a wearer's pelvic region creating a vertical overlapping double flap fly and a stretchable material inlay is concealed by the vertical overlapping double flap fly, wherein the stretchable material inlay has a tensional opening.

Other and further features and advantages of the present invention will be apparent from the following descriptions of the various embodiments. It will be understood by one of ordinary skill in the art that the following embodiments are provided for illustrative and exemplary purposes only, and that numerous combinations and modification of the elements of the various embodiments of the present invention are possible.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of embodiments of the present invention, reference is made to the following Detailed Description, which is to be read in association with the accompanying drawings, wherein:

FIG. 1 is a front view of an exemplar undergarment system with a double flap fly in accordance with an embodiment of the invention;

FIG. 2 is a front view of the undergarment system of FIG. 1 with the double flap fly open;

FIG. 3 is an inside sectional view of an exemplar undergarment system in accordance with an embodiment of the invention; and

FIG. 4 is a front view of another undergarment system in accordance with an embodiment of the invention.

### DETAILED DESCRIPTION OF PREFERRED EMBODIMENTS

The embodiments of the present invention are described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as systems, or devices. The following detailed description should not to be taken in a limiting sense.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” The term “coupled” implies that the elements may be directly connected together or may be coupled through one or more intervening elements.

As illustrated in the figures, the novel prophylactic undergarment system is designed to look like, and intend to be worn as, everyday underwear. Although the embodiments depict how a tensional opening can be sewn or assembled into a double layered piece of material that is then sewn (or otherwise assembled) into the crotch region of the undergarment and how such a system can be easily accessed and concealed by an overlapping double flap fly, the embodiments are not limited to boxer shorts. Such a system may be incorporated into all types of men's bottom wear, which include: underwear, boxers, boxer briefs, briefs, underpants, undershorts, undergarments, underclothes, undies, skivvies, drawers, shorts, pants, sweat pants, leggings, wide trousers, breeches, buckskin breeches, riding breeches, kilts, thermals, long johns, long underwear, unitards, diving suits, swim trunks, swimsuits, thongs, jockstraps, board shorts, gym shorts, jogging shorts, jockey shorts, spandex shorts, briefs, bikini briefs, tanga briefs, etc. These common designs also provide a good level of coverage for the wearer from skin to skin contact and fluid transfers during sexual activity.

The embodiments provide for a novel highly effective disease preventing, condom-retaining device with a built-in sexual enhancement mechanism. In a preferred embodiment, the sexual enhancement mechanism is a “cock-ring.”

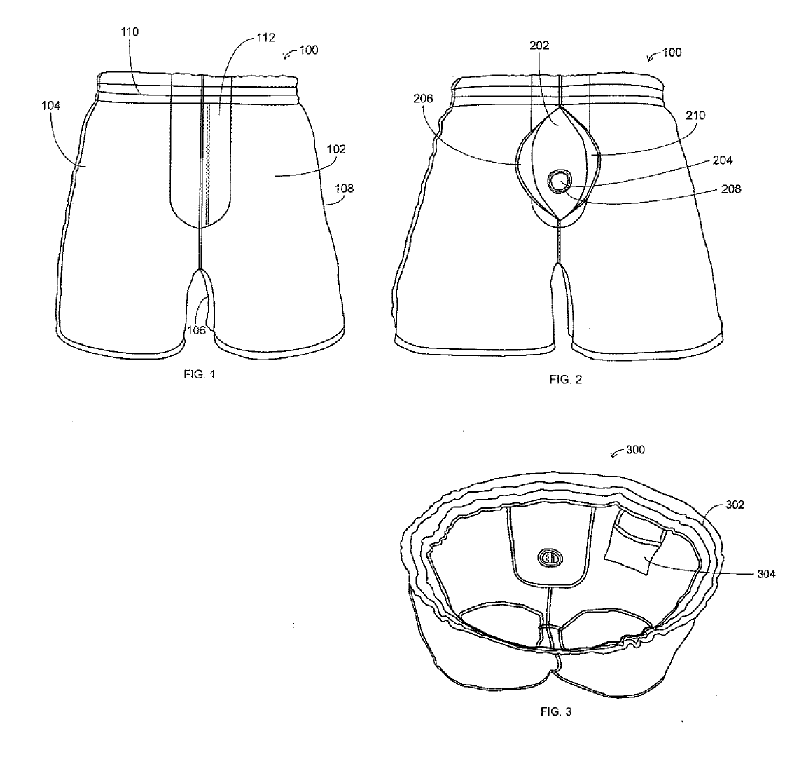

FIG. 1 depicts a front view of an undergarment system 100. The undergarment system 100 has a flexible outer casing 102. with a non-permeable inner surface and an outer surface. The flexible outer casing 102 may be constructed from a single textile having a different surface on the inner side than the outer side. Alternatively, it may be constructed from multiple textiles having the same surfaces or different surfaces or may be constructed from a single textile having the same surface on the inner surface and outer surface. The textile used for the undergarment 100 may vary in the type depending on the intended function of the undergarment 100. For example, some models may be designed to prevent just skin-to-skin contact, while others may be designed to prevent fluid transfer, and yet some may be designed to be disposable or designed for limited or single use. The materials may be non-permeable on all surfaces or non-permeable on select surfaces. Preferably, the materials used are of sufficient strength and quality that the undergarment system may be washed or otherwise laundered and reused at least 10 times before the non-permeable surface is compromised. Furthermore, the undergarment 100 may be comprised of a combination of materials. For example, in one embodiment the front and the back of an undergarment are comprised of two entirely different materials. In another embodiment, fluid proof material is used on the front where sexual contact takes place and the back may be comprised of a different material because fluid proof protection may not be needed in the latter location. In one embodiment, materials that possess both stretch and fluid proof properties are implemented only in the crotch section of the undergarment

The flexible outer casing 102 may be constructed from multiple panels or from a single panel. Regardless of the number of panels, the flexible casing 102 includes front 104, rear 106 and lateral 108 aspects that define the undergarment 100. Although referred to in the singular, the front, rear and side aspects may be constructed from a single piece of textile or multiple pieces. The upper end of the undergarment 100 has a flexible waistband 110. The flexible waistband 110 may include elastic, drawstrings, or mechanical fasteners or any other construction that allows manipulation of the waistband size. The front aspect 104 includes a double flap fly 112. The double flap fly 112 is comprised of two tabs, which extend from the front aspect 104 of the undergarment 100. Preferably the double flap fly is centered over the portion of the undergarment 100 covering the pelvic region. In one embodiment, the tabs of the double flap fly 112 are the same size; in alternate embodiments, the flaps may be of different sizes. In a preferred embodiment, the flaps over lap by at least one inch. In another embodiment, the fly flaps are preferably at least 5 inches in length. Preferably the double flap fly is vertical in alignment however this is not a limitation on the embodiments of the present invention, the double flap fly may be arranged horizontally or at an angle.

FIG. 2 illustrates a front view of the undergarment 100 with the double fly flap 112 open. When open the double fly flap 112 does not expose a wearer's body but rather, a stretchable material inlay 202 assembled behind the double flap fly 112 and attached to the front aspect 104 of the undergarment 100. Although described as a stretchable material this is not intended to be a limitation on the embodiments of the present invention, it is contemplated that embodiments of the present invention may include other types of inlays including but not limited to latex, polypropylene, polyurethane, rubber, spandex, lycra, and rayon. The stretchable material inlay 202 has a tensional opening 204. In its un-deformed shape, the tensional opening 204 is preferably a circular hole. The tensional opening 204 is designed to accommodate the wearer's penis. Alternatively, the tensional opening 204 may be designed to accommodate a dildo or other object for sexual stimulation.

A band 208 surrounds the tensional opening 204. The band 208 is preferably a ring. The ring 208 may be constructed from a variety of materials. Preferably the ring 208 is constructed from a material that has elastic or resilient properties giving the hole sustained resilience over repeated use for example, elastic, latex, or rubber. Alternatively, the ring 208 may be constructed from leather, metal, or other materials used for sexual performance enhancement mechanisms. In another alternative, the ring 208 is constructed such that its size may be manually adjusted. Non-limiting examples include a ring that has a buckle or notch system, a drawstring, a hook and loop system i.e., velcro. The ring 208 may act as a sexual performance enhancement device; the ring 208 may also or alternatively act as a condom-retaining device.

The ring 208 is sewn or otherwise assembled (i.e., heat set, glued, manually fastened, assembled using ultrasonic fusion, or any other means) into the material inlay 202. The material inlay is a single, or multi layered, piece of soft, stretchable, fluid proof, nonporous, stain resistant, high performance medical material or textile or any other wearable material. In other embodiments, the ring is removable and a wearer may replace it with alternate sizes of rings or rings that have varying capabilities. The double flap fly 112 comprises a top flap 206 that overlays a bottom flap 208. Preferably, the top flap 206 overlays the bottom flap 208 by approximately 1.1 inches or less. Although depicted as the left side of the double flap fly 206 overlapping the right side of the double flap fly 208, this is not intended to be a limitation on the embodiments and the double flap fly 112 may be configured to overlap right to left, or may not overlap at all but rather the two fly flaps may merely lie adjacent to one another. Preferably the median overlapping of the double flap fly 112 aligns vertically with the wearer's anatomical center, such that when pulled apart or open, the double flap fly 112 creates an opening approximately 5 inches in length (from top to bottom) and 6 inches in width (from left to right). More preferably, the double flap fly allows for an opening of at least 3 inches in vertical length and 3 inches in horizontal width when the fly is opened, i.e., the top flap 206 and the bottom flap 208 are separated. However, embodiments of the invention include any comparable adaptation or variations of the above-described double flap fly (such as size, positioning, and/or fastening variants, etc.). Similarly, embodiments of the invention include any adaptations or variations to the tensional opening, for example its shape, size (both stretched and unstretched) and placement. Furthermore it is contemplated within the scope of the embodiments of the invention that the size of the waistband may vary both is circumferential length and width, to accommodating varying sizes and comfort factors of the wearer.

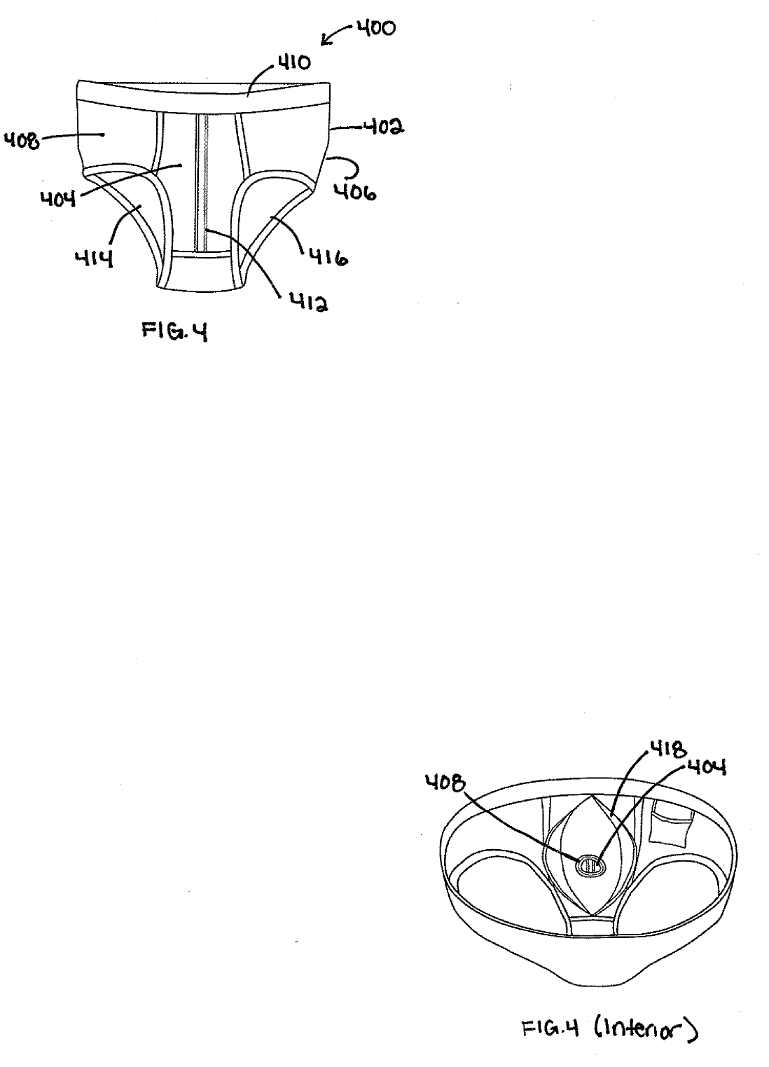

FIG. 3 illustrates the inside view of an undergarment 300 in accordance with an embodiment of the invention. Coupled to a flexible adjustable waistband 302 is a pouch 304 for holding a condom. The pouch 304 hangs below the interior waistband 302 off the right hip. Preferably, the pouch is located within six inches of the centerline of the pelvic region. By being positioned off the waistband 302, the pouch 304 hangs comfortably off the waistband 302 preventing the condom from damage due to excessive body heat. In addition, the pouch 304 itself also helps protect the condom from sustaining any physical damage. While described as located off the right hip, this is not intended to be a limitation on embodiments of the invention and the pouch 304 may be located anywhere along the waistband 302. Alternatively, the pouch 304 may be coupled to the outside, a side seam or otherwise positioned in the undergarment. Preferably, but not essentially, the pouch 304 is cut to size so that it snugly fits and effectively holds a condom without the use of any snaps or flaps. Preferably, the pouch 304 is fabricated from a single piece of textile or any other wearable material measuring approximately 3 inches wide and 8 inches long. The bottom 3 inches is folded up and over itself. The edges are folded and sewn, or otherwise assembled, in effect, creating a condom pouch approximately 2½ inches by 2½ inches, leaving an extra 2 inches of loose textile at the top. Of the 2 inches, 1 inch is sewn, or otherwise assembled, into the interior waistband and the other inch allows the condom pouch to dangle 1 inch below the interior waistband. As a result in the preferred configuration, the pouch 304 is discreetly positioned out of sight and coupled to the undergarment by the small piece of textile, or any other wearable material that extends off the top of the pouch consequently the rest of the pouch hangs freely just below waistline so that the pouch could be easily cut-off, if so desired.

In an embodiment, the material used for the undergarment may contain a specialized marking system that changes color over time in order to provide the wearer a visual indicator as to when the undergarment needs to be replaced. The undergarment itself may be constructed from the specialized material having a visual indicator or the garment may have a liner made from the specialized material or the condom pouch may be made from the specialized material, or a strip may be attached to the inside or a label on the outside of the garment may be constructed from the specialized materials. In other embodiments, the visual indicator could be an accoutrement other than a specialized material, non-limiting examples include: a digital indicator showing the number of times the garment has been submersed in water; an LCD or other display indicating the number of times the garment has been used or the number of times it has been cleaned, a meter or other gauge displaying use. The visual indicators could be supported by a battery, could be mechanically wound during use or any other means to provide the necessary support for the digital indicator or other display.

Preferably, the visual indicator is located on the outside of the undergarment so that both the wearer and the partner may see the indicator as each would be interested in the level of protection provided by the undergarment system.

FIG. 4 illustrates another embodiment of the invention. The undergarment system 400 has a flexible outer casing 402 with a non-permeable inner surface and an outer surface. The flexible outer casing may be constructed from a single textile having a different surface on the inner side than the outer side or the same surface on the inside and outside. Similarly, the textures may vary from inner surface to outer surface or may be the same. Alternatively, the undergarment may be constructed from multiple textiles. The flexible outer casing 402 may be constructed from multiple panels or from a single panel. Regardless of the number of panels, the flexible out casing 402 includes front 404, rear 406 and lateral 408 aspects that define the undergarment 400. The upper end of the undergarment 400 is constructed with a flexible waistband 410. The waistband 410 may include elastic, drawstrings, or mechanical fasteners or any other means that allows the waistband size to be manipulated. The lower end of the undergarment 400 has two opening 414, 416 through which a wearer's legs extend. The two opening 414, 416 are flexible at the lower end. The openings 414, 416 may include elastic so that the openings 414, 416 stay in contact with the wearer's legs thereby limiting the movement of the undergarment from the wearer's body and providing additional protection from fluids or other disease spreading elements.

The front aspect 404 includes a fly 412. The fly 412 does not expose a wearer's body but rather is a stretchable material inlay 402 with a tensional opening 404. The tensional opening 404 (FIG. 4 (Interior)) is designed to accommodate the wearer's penis. Alternatively, the tensional opening 404 may be designed to accommodate a dildo or other object for sexual stimulation.

A band 408 surrounds the tensional opening 404. Preferably, the band 408 is a ring. The ring 408 may be constructed from a variety of materials. Preferably the ring 408 is constructed from a material that has elastic or resilient properties giving the hole sustained resilience over repeated use for example, elastic, latex, or rubber. Alternatively, the ring 408 may be constructed from leather, metal, or other materials used for sexual performance enhancement mechanisms. Less preferably, the ring 408 is constructed such that its size may be manually adjusted. In a single use embodiment of the undergarment 400, the ring 408 need not be required to sustain resilience over repeated use. The tensional opening 404 and ring 408 are designed to accommodate penises of all sizes. Overall the opening and ring may vary from 0.75-3.5 inches. For example, in un-stretched condition, they may be approximately 0.75 inch in diameter, and when fully stretched the hole expand to 3.5 inches in diameter. These are not intended to be limitations on the size of the unstreched and stretched opening but merely represented a preferred range, it is understood that the unstreched opening may be less than 0.75 inches and stretched may exceeed 3.5 inches. The tensional (stretchable) hole may be produced to possess variable sizes and elasticity properties for those wearers who desire an opening and ring that are more specific to their needs. However, in all embodiments preferably the wearer uses an embodiment wherein the diameter of the opening and ring is smaller in its un-stretched condition than the diameter of wearer's penis and must therefore be stretched outward in order to fit a penis through it. The sizing is necessary to form a constrictive ring around the condom-covered penis, that when fully inserted into the stretchable ring of the undergarment then snugly rests up against the latex ring at the base of the condom, holding the condom in place. This results in forming an impervious seal around the condom-covered penis that keeps the latex ring at the base of the condom on the interior side of the undergarment while the rest of the condom-covered penis protrudes though to the exterior side of the undergarment. In embodiments of the invention, the constrictive nature of the ring may also help maintain prolonged erections by slowing the flow of blood from the erect penile tissue.

The ring 408 is sewn, or otherwise assembled, into a single (or multi layered) piece of soft, stretchable, fluid proof, stain resistant, anti-bacterial, high performance medical material or any other wearable material that comprises the stretchable material inlay 402. Although the material inlay is described as having several properties, it is contemplated within the scope of the embodiments of the present invention that not all of the properties are required and embodiments may have fewer than all properties. Preferably, the material is also noise free and has anti-static properties. In one embodiment, the stretchable material inlay is a single piece of a single layer material and the ring 408 is assembled by weaving the ring 408 through the fibers in the material. In another embodiment, the stretchable material inlay is a single piece of a single layer material and the ring 408 is assembled by affixing the ring 408 to the material. In a single use embodiment of the undergarment 400, the undergarment may be constructed from a disposable textile having at least one side coated with a non-permeable formulation.

In another embodiment, the undergarment 400 may optionally include an interior double flap fly 418 (FIG. 4 (Interior)). The interior double flap fly 418 is comprised of two tabs, which extend from the inner surface of the front aspect of the undergarment 400. Preferably the double flap fly is centered over the tensional opening of the undergarment 400. The interior double flap fly covers the tensional opening and helps make the undergarment more comfortable to the user. The double flap fly may be made from any of the materials used for the undergarment or of entirely different materials with increased softness and cushioning. In another embodiment, a soft or cushioned material is fabricated on the inner surface of the stretchable material inlay 402. In this embodiment, an opening is constructed in the soft or cushioned material such that the opening aligns with the tensional opening 404 and ring 408 in stretchable material inlay 102. In another embodiment the interior double flap fly is made of materials that are fluid absorbent, such that the materials collect any moisture associated with male urinary incontinence or pre-ejaculatory emissions that may occur prior to engaging in sexual activity.

In another embodiment, the undergarment is constructed in a manner similar to a men's bathing suit. In such an embodiment, the undergarment has an outer casing resembles a pair of shorts and an inside liner, resembling a brief. The outer casing has a fly flap which when open exposes the inside liner. The front crotch area of the inside liner has a tensional opening with a band, as described above in relation to the stretchable material inlay. In this embodiment, the outer casing may be constructed from any textile as the inner lining provides the protection. One advantage of this embodiment is that the inner lining is constructed in similar fashion to a brief, therefore even if a portion of the outer casing rise up along the wearer's legs, the wearer's genital area is protected by the inner lining.

In use, embodiments of the invention provide novel systems for reducing skin-to-skin contact and the exchange of body fluids, which prevent the transmission of sexual diseases commonly passed or “shed” around unprotected genital areas beyond typical condom coverage. A person wearing an embodiment of the undergarment system, after removing a condom from the built-in pouch, unwraps the condom and fully applies it to the erect penis. The wearer then places his penis through the tensional opening and through the open double flap fly, never having to remove the undergarment. The penis with the condom is the placed through tensional opening until the elastic ring assembled in the opening abuts the latex ring at base of the condom. The elastic ring assembly prevents the condom from slipping off while in use or upon extraction from the wearer's partner so that the condom is secured under the elastic ring and does not slip. In addition to retaining the condom in place and increasing its disease preventing capabilities, the ring acts as a “cock ring” enhancing the wearer's sexual performance capabilities.

After use the wearer first removes the undergarment system. After the undergarment system is removed, the wearer next removes the condom using care to minimize contact with the condom. After the undergarment system and condom are removed, the wearer should wash his body with an antibacterial soap. Upon removal the condom should be discarded. The undergarment system should be placed in the laundry for washing or discard if disposable. The undergarment system should be preferably washed in warm to hot water using conventional detergent. In no instance should the undergarment system be used a second time without first being laundered.

Embodiments of the invention having the double flap fly are designed to conceal the stretchable penis-sized hole that lies behind the double flap fly to help subvert some of the anticipated social stigmas that may be associated with the initial use of the embodiments. This conventional and familiar design helps evade any judgment subjected upon its wearer due to the suggestive nature of the product and any misinterpreted implications it may bring.

As noted previously the forgoing descriptions of the specific embodiments are presented for purposes of illustration and description. They are not intended to be exhaustive or to limit the invention to the precise forms disclosed and obviously many modifications and variations are possible in view of the above teachings. The embodiments were chosen and described in order to explain the principles of the invention and its practical applications, to thereby enable those skilled in the art to best utilize the invention and various embodiments thereof as suited to the particular use contemplated. It is intended that the scope of the invention be defined by the claims and their equivalents.

1. A prophylactic undergarment system comprising:
a flexible outer casing having front, rear and lateral aspects defining an undergarment with a flexible waistband;
the front aspect, having two exterior panels, equidistantly positioned and centered over a wearer's pelvic region creating a vertical overlapping double flap fly; and
a stretchable material inlay concealed by the vertical overlapping double flap fly, wherein the stretchable material inlay has a tensional opening comprised of a resilient material.

2. The undergarment system of claim 1, further comprising:
a containment pouch coupled to the flexible waistband, wherein the containment pouch is sized so that it may securely hold at least one condom.

3. The undergarment system of claim 2, wherein the containment pouch coupled to the flexible waistband is located on the inside of the undergarment system and within 6 inches of the tensional opening.

4. The undergarment system of claim 1, wherein the undergarment is a men's boxer, brief, or boxer brief.

5. The undergarment system of claim 1, wherein the tensional opening is an elastic ring having variable stretch capabilities such that the opening ranges between a 0.75 inch wide opening to a 3.5 inch wide opening.

6. The undergarment system of claim 5, wherein the stretchable material inlay is comprised from at least two pieces of material assembled such that the elastic ring lies between the at least two pieces of material.

7. The undergarment system of claim 5, wherein the tensional opening is a sexual performance mechanism.

8. The undergarment system of claim 5, wherein the tensional opening is a condom-retaining device.

9. The undergarment system of claim 5, wherein the tensional opening is manually adjustable.

10. The undergarment system of claim 1, wherein the flexible outer casing; the exterior panels and stretchable material inlay further comprises a soft material having anti-static and sound resistant properties.

11. The undergarment system of claim 1, wherein the front aspect, (the two exterior panels are described as the front aspect in claim 1) and the stretchable material inlay are comprised of a stain-resistant material.

12. The undergarment system of claim 1, wherein the front aspect, and the stretchable material inlay further comprise a non-permeable inner surface and an outer surface.

13. The undergarment system of claim 12, wherein the undergarment system can be washed and reused at least 10 times before the non-permeable surfaces are compromised.

14. The undergarment system of claim 1, further comprising a visual indicator on the outer surface of the undergarment to specify the functionality status of the garment.

15. The undergarment system of claim 1, wherein the double fly flap comprises a top flap that overlays a bottom flap by not more than 1.1 inches.

16. The undergarment system of claim 1, wherein the top flap and the bottom flap create an opening of at least 3 inches in vertical length and at least 3 inches in horizontal width when separated.

17. The undergarment of claim 1, further comprising two interior panels, equidistantly positioned and centered over a wearer's pelvic region creating a vertical overlapping double flap fly.

18. The undergarment system of claim 17, wherein the two interior panels are comprised of a soft, fluid absorbent material.

19. A prophylactic undergarment system comprising:
a flexible outer casing comprising front, rear and lateral aspects defining an undergarment with a flexible waistband; and
the front aspect having a stretchable material inlay located equidistance from the lateral aspects wherein the stretchable material inlay has a tensional opening comprised of a resilient material.

20. The undergarment system of claim 19, wherein the undergarment system is comprised of materials intended for single use.
